(19) United States
(12) Patent Application Publication
(10) Pub. No.: US 2007/0072245 A1
(43) Pub. Date: Mar. 29, 2007

(54) Method and means for the determination of defined states or modifications in the mucus of the uterus or in the epithelium of other organs

(76) Inventors: Henry Alexander, Leipzig (DE); Gerolf Zimmermann, Leipzig (DE)

Correspondence Address: GUDRUN E. HUCKETT DRAUDT, LONSSTR. 53, WUPPERTAL, 42289, DE

(21) Appl. No.: 10/559,406

(22) PCT Filed: Jun. 4, 2004
(86) PCT No.: PCT/DE04/01210

§ 371 Date: May 30, 2006

(30) Foreign Application Priority Data
Jun. 6, 2003 (DE) 10325639.3
Jun. 6, 2003 (DE) 10325638.5

(51) Int. Cl. G01N 33/567; C07K 16/26; C07K 14/59

(52) U.S. Cl. 435/7.2; 530/388.24; 530/397

(57) ABSTRACT

The invention relates to a method and means for the determination of defined states or modifications in the mucus of the uterus or in the epithelium of other organs, especially for the diagnosis of pregnancy and diagnosis of embryo-implantation. In a biological sample, the concentration of human endometrial chorionic gonadotropin (eβhCG/ehCG) and/or non-trophoblastic hCG (hCG type I, β6, β7) is determined in a specific manner, wherein the hCG (hCG type II, tβhCG) which is to be trophoblastic is different. The invention also relates to the sequence of the β sub-unit of human endometrial chorionic gonadotropin of eβhCG, and anti-bodies which are specifically different from eβhCG/ehCG and hCG type II, tβhCG, and to a test kit for carrying out said method.

The invention relates to method and means for determining defined states or modifications in the mucous membrane of the uterus or in the epithelium of other organs, in particular for diagnostics of pregnancy and its dysfunctions as well as diagnostics of the onset of labor and for determining optimal implantation conditions within the uterus, and the diagnostics of physiological and pathological epithelium states (carcinoma). Field of application is medicine, in particular, gynecology with the specific areas of reproductive medicine and obstetrics.

Human chorionic gonadotrophin (hCG) is a glycoprotein and is comprised of two subunits αhCG and βhCG that are non-covalently bonded (1). For the subunit βhCG a gene is known (chromosome 6q21.1-q 23). For the subunit βhCG there are 7 genes β8, β7, β6, β5, β3, β1 and β2 known (chromosome 19q13.3).

During pregnancy, large amounts of hCG dimer and free βhCG and βhCG molecules are formed by the trophoblast in the uterus and secreted into the blood. Embryonic trophoblastic tissue expresses almost exclusively hCG β5, β8, and β3. These βhCG subunits are therefore also referred to as trophoblastic βhCG (tβhCG) or type II βhCG.

However, in some non-trophoblastic tissues, hCG or its subunits are also expressed in minimal quantities (2-6). Non-trophoblastic tissue, for example, mamma tissue, lung tissue, prostate tissue, bladder tissue, colon tissue, express almost exclusively hCG β7 and β6. These βhCG subunits are therefore referred to as non-trophoblastic βhCG or type I βhCG (7).

In the blood of healthy humans who are not pregnant hCG concentrations of hCG up to 1,000 pg/ml and of βhCG up to 100 pg/ml are therefore observed (8, 9). Higher βhCG serum values indicate a gonadal or non-gonadal tumor and indicate an unfavorable prognosis as described in connection with lung carcinoma, bladder carcinoma, prostate carcinoma, colon carcinoma, kidney cell carcinoma or mamma carcinoma (5, 10-14).

while the subunits of the type II βhCG (β5, β8, and β3) contain at position 117 (exon 3) of the amino acid sequence an aspartate (Asp, D), the type I βhCG (β7 and β6) contains alanine (Ala, A) at position 117. The βhCG gene β6 is an allele of of β7 with differences in the 5=-non-translating sequence of the promoter gene (exon 1) and in the translating sequence (exon 2) of the βhCG subunit. Only the genes β8, β7, β6, β5, and β3 code and express a βhCG protein molecule of 145 amino acids (exon 2 and exon 3). The genes hCG β1 and β2 can be transcribed also in some tissues but code a protein of only 132 amino acids with different sequences relative to βhCG (15-17).

The hCG molecule is well-characterized by known standard preparations of up to now 24 monoclonal hCG antibodies of the International society of Oncodevelopmental Biology and Medicine (ISOBM) that recognize different defined epitopes on the βhCG subunit (α1-α7, n=7), on the βhCG subunits (β1-β9, n=9), on the βhCG core fragment (cfβ10-cfβ13, n=4), and also conformation-dependent epitopes of the intact αβ heterodimer (αβ1-αβ4, n=4) specifically and with high epitope affinity (18-20).

The antibodies recognize preferred epitopes in the spatial amino acid arrangement of the hCG molecule, i.e., of its tertiary and quaternary structure (17; FIG. 2 and FIG. 3 in 18). For this reason, for the preparation of hybridomas for the formation of the aforementioned monoclonal ISOBM antibodies WHO reference hormone preparations of the hCG, respectively, βhCG of the International Federation of Clinical Chemistry (IFCC) are used as much as possible as adequate antigenic determinants. Exceptions are the βhCG epitopes β8 (AS 137 to 144) and β9 (AS 109 to 116) of the C-terminal end (CTP) as well as a portion of the βhCG epitope β3 (AS 1 to 10) of the N-terminal end of βhCG that represent an immunological antigen potential (21, 22) at the edge of the surface of the hCG dimer in cystine knot structure and substantially unaffected by the tertiary structure. This linear βhCG epitope sections about AS 1-16, AS 108-123, and AS 137-144 are recognized by monoclonal βhCG antibodies that are produced by the method of carrier-bonded synthetic peptides as antigenic determinants of the ISOBM antibodies (18, 19, 21, 22).

For the antigen region β3, β8, β9 of the βhCG molecule (FIG. 3 in 18) ISOBM βhCG antibodies have already been prepared; they are based on the known amino acid sequence of the trophoblastic (or placenta) βhCG subunit. Especially the amino acid sequence about 108-123 of the C-terminal end of βhCG (βhCG-CTP) characterized as epitope β9 shows a high antigenicity (21). To a somewhat lesser degree, this also holds true for the amino acid sequence about 1-16 as a part of the epitope β1 (23). Antibodies generated with synthetic peptides against these peptide sequences recognize specifically the native trophoblastic βhCG subunit (18, 19, 21, 23, 24).

In the past, the different studies have been undertaken with the goal to detect by means of semi-quantitative methods (5, 12, 13, 25) βhCG transcripts in different normal and neoplastic tissues of non-trophoblastic origin. These methods show that βhCG is transcribed in normal placenta (26), healthy testes (6), but also neoplastic testes (27) and neoplastic bladder tissue (28). However, in these studies no differentiation is being made between type I βhCG and type II βhCG.

For this purpose, different test kits have been proposed that function with different epitope specificity (29) and that enable the determination of the trophoblastic total molecule αβhCG (total hCG) or of the individual subunits of the molecule βPhCG and αhCG (18). Especially for pregnancy tests and diagnostics of hydatid moles and choriocarcinoma methods have been developed in which the heterodimer trophoblastic total molecule hCG alone (total thCG) or the sum with the free subunit of the trophoblastic βhCG (total tβhCG plus tβhCG) or the free tβhCG subunit alone is determined. The heterogeneity of the trophoblastic hCG in biologic material (intact αβ heterodimer, free αhCG, free tβhCG, fβhCG core fragment, nicked thCG, nicked βhCG) relative to bonding on the respectively employed antibodies, make a precise determination and standardization of a possible detection method for the epithelial hCG more difficult. An additional heterogeneity of the tβhCG determination in the secretory cycle phase and early pregnancy can occur to a minimal degree also between the four native hyperglycolyzed or desialylated hydrocarbon side chains of the C-terminal end (CTP) of tβhCG (amino acid 120 to 145) as they have been observed in differentiated forms in the early to middle stages of a pregnancy and for choriocarcinoma in trophoblastic βhCG (31, 33-37).

The pregnancy tests known so far have the disadvantage that they often render false positive results.

The currently known pregnancy tests can evaluate only unsatisfactorily reduced hCG concentration measurements during the extra uterine pregnancy (ectopic pregnancy) or hCG titer in female patients after IUD insertion under the aspect of changed uterine secretion behavior (38, 39).

The phenomenon that a pregnancy test in the blood is positive for hCG, even though no pregnancy or no tumor in the genital tract is present, is referred to as a phantom hCG. The current discussion of phantom hCG values is based on the fact that this phenomenon is derived from abnormal interaction between the test and irregular antibodies contained in the blood sample of the female patient (54).

It is an object of the invention to provide method and means that enable detection of defined states or modifications in the mucous membrane of the uterus (endometrium, decidua) but also in the epithelium of other organs. The method and the means should enable in particular the determination of optimal implantation conditions in the uterus and a reliable diagnostics of a pregnancy and their dysfunctions as well as the beginning of the birthing process.

The invention is based on the scientific recognition that in the endometrial tissue and the decidual epithelial βhCG subunits are expressed that differ in several amino acid positions from the known trophoblastic βhCG subunits.

The nucleotide sequence and protein sequence for the endometrial βhCG subunits is represented for the first time in SEQ ID No. 7 and SEQ ID No. 10 (eβhCG or endo).

The βhCG subunits expressed in the endometrial and the decidual epithelium are referred to in the following as endometrial βhCG (eβhCG). Our results indicate that the eβhCG represents an endometrial variant of the subunits β7 and β6, while trophoblastic βhCG is formed exclusively of the subunits β5, β8, and β3.

The following differences of the eβhCG to the known trophoblastic βhCG (tβhCG) have been found:

One variant of aspartate (tβhCG) in the amino acid position 117 of the C-terminal end of βhCG (βhCG-CTP) to alanine (eβhCG).

Further variants were found in position 2 and position 4 of the exon 2. The trophoblastic βhCG (tβHCG) has at position 2 lysine and at position 4 proline. The endometrial βhCG (eβhCG) has at position 2 arginine and at position 4 methionine.

In the following, the term endometrial βhCG (eβhCG) is to be understood as a βhCG that has at least one of the aforementioned variants.

FIG. 1 shows an alignment of the sequence of the eβhCG (endo) with the sequences of the trophoblastic βhCG subunit tβhCG β5 and the known non-trophoblastic βhCG subunits β6 and β7 as well as the pituitary βLH β4 (β subunit of the luthetic hormone).

The nucleotide sequence of the endometrial eβhCG (SEQ ID No. 7) differs also from the known non-trophoblastic βhCG subunits β7 (SEQ ID No. 5) and β6 (SEQ ID No. 6), in particular in the promoter gene of the exon 1 but also in exon 2 at the expression location of the amino acid positions 2 and 4 (FIG. 1). In the amino acid sequence (SEQ ID No. 10) resulting from gene expression, the endometrial eβhCG presents itself as a variant of the epithelial type I βhCG of the βhCG β7 protein (SEQ ID No. 9) with three different amino acids at positions 2, 4 and 117 between the endometrial or decidual eβhCG and the conventional trophoblastic tβhCG (SEQ ID No. 8). The amino acid sequence of the endometrial eβhCG (SEQ ID No. 10) differs significantly (FIG. 1) relative to the sequence of the pituitary βLH β4 (SEQ ID No. 11).

Furthermore, the invention is based on the scientific recognition that the reason for the false positive results known in the case of prior art tests is that they cannot differentiate between the trophoblastic secretion output of the embryo in the form of the tβhCG and the secretion output that result from the secretion transformation and uterine decidua formation of the endometrium as well as during epithelial differentiation (eβhCG).

Method:

Based on this recognition, the object is solved according to the invention by a method for determining defined states or modifications in the mucous membrane of the uterus or in the epithelium of other organs in that in a body liquid sample and/or tissue sample the concentration of endometrial βhCG or non-trophoblastic βhCG is determined specifically. The term specific eβhCG determination is to be understood in that a differentiation is made between endometrial βhCG and trophoblastic βhCG.

The determination of concentration is realized preferably in a sample (in the form of secretions, perfusion liquid, cells or tissue) of peripheral blood, serum, menstrual blood, lochia, amniotic fluid, urine, saliva, eye chamber fluid as well as secretions of the urogenital tract (including uterus, cervix, vaginal samples), of the gastrointestinal (including mucous membrane of the mouth) and of the respiratory tract as well as of the central nervous system (incl. liquor).

Preferably, in the method according to the invention, in addition to the concentration of endometrial βhCG or non-trophoblastic βhCG also the concentration of trophoblastic βhCG (tβhCG), total βhCG or total hCG is determined.

The determination of trophoblastic βhCG (tβhCG ), total βhCG, or total hCG is carried out preferably according to known methods (18, 19, 21, 22, 23, 24, 29, 30, 31, 54).

Since these methods known in the art do not differentiate between tβhCG and ehCG, the specific determination according to the invention of eβhCG for the first time also enables a determination whether the tβhCG determined with these tests is indeed tβhCG and how high the proportion of tβhCG and eβhCG is.

The proportion of tβhCG results from the concentration of the total hCG/βhCG or the total hCG minus the measured eβhCG or the non-trophoblastic βhCG.

Advantageously, the method according to the invention is suitable in particular for diagnosis of the readiness (receptivity) of the mucous membrane of the uterus for a fertilized egg and enables thus the determination of optimal implantation conditions in the uterus.

It was found that the expression of eβhCG in the mucous membrane of the uterus (endometrium) is required in order to enable the successful implantation of the fertilized egg. The beginning of the eβhCG or of ehCG is an indication for the receptivity of the mucous membrane of the uterus for a fertilized egg.

The diagnosis of the receptivity of the mucous membrane of the uterus (implantation condition) for a fertilized egg is carried out preferably prospectively in that from a female patient in the early luteal phase tissue is removed from the endometrium or from the cervical mucous membrane (mucous membrane of the mouth), or a secretion of the vagina, the cervix, or the uterus, or serum, plasma and peripheral blood is removed; in this sample, the non-trophoblastic or endometrial βhCG concentration is determined. Based on the level of the determined expression it is then possible to draw conclusions in regard to the current receptivity of the uterus for an embryo or a prognosis for the following cycle.

For this purpose, preferably several days after ovulation cells are removed by means of a mini catheter from the uterus cavity, by means of a cotton swap from the cervical channel, or by means of a wooden spatula from the mucous membrane of the mouth or peripheral EDTA or heparin blood is taken. In the taken cells the non-trophoblastic or endometrial βhCG concentration is determined.

For the prospective diagnostics of the embryo receptivity in the early secretion phase of the actual cycle, preferably tissue samples of the endometrium, the endocervix, the mucous membrane of the mouth or other select epithelia as well as cervical/vaginal secretions or endometrial secretions after smear or as a perfusate are examined in order to determine the quality of the secretory transformation to be expected and receptivity of the endometrium (for example, for the determination of an embryo transfer after in-vitro fertilization in a hormonally stimulated cycle).

Based on the information of implantation conditions in the preceding cycle, a prognosis for the implantation conditions, i.e., receptivity of the uterus for the fertilized egg or an embryo, in the following cycle can be made.

A further preferred use of the method is therefore the application for retrospective diagnostics of the receptivity of the mucous membrane of the uterus. The term retrospective implantation diagnostics in the context of the present invention is to be understood as the detection of the secretory transformation of the endometrium of the preceding cycle in order to make projections for the receptivity of the subsequent cycle. They exhibit an undisturbed fallopian tube/uterus relationship that is timely and functionally appropriate. The method according to the invention therefore can supplement or replace the invasive method of a Pap smear in regard to its information contents.

By means of the evaluation and quantification of the specific epithelial endometrial hCG secretion (ehCG) in bodily fluids and cell (tissue) homogenates of the early, middle and late secretion face of the menstrual cycle, optimal implantation conditions as well as possible fertilization dysfunctions can be determined prospectively as well as retrospectively under the aspect of endometrial diagnostics and therapy control.

In the retrospective diagnostics of the receptivity of the mucous membrane of the uterus, the same method as for a prospective (preparatory) implantation diagnostics can be carried out in principle. Preferably, the analysis of the βhCG concentration is carried out in a sample of menstrual blood or a sample of cells contained in the menstrual blood.

In the menstrual blood, sufficient cells of the endometrium are present that enable a determination of the βhCG concentration.

The advantage of the retrospective diagnostics in the menstrual blood relative to the afore described prospective method resides in that it is not invasive. Neither peripheral blood nor a tissue sample of the uterus must be taken. In spite of this, with this method a timely and functionally appropriate conversion of the endometrium in the secretion phase of the cycle can be detected which is at the same time an expression of an undisturbed regulation function at the level of hypothalamus/pituitary gland, ovaries, and uterus.

Menstrual blood like peripheral blood can be used, after centrifugation for the purpose of separating cells and stroma, for the direct measurement of endometrial βhCG secretion with specific antibodies in the ELISA test. The menstrual blood is taken after a spontaneous cycle, after hormone therapy, after in-vitro fertilization (IVF), and embryo transfer (ET) without successful implantation as well as in case of preparatory diagnostics of the cycle in the case of female patients wanting children and female patients with gynecological diseases such as myoma, endometriosis, endometrial carcinoma and cervix carcinoma. The parallel harvesting of peripheral blood simultaneous to heparin blood or serum is mandatory for excluding an increased unspecific serum hCG value.

When the concentration of the eβhCG or non-trophoblastic βhCG in menstrual blood is increased relative to the concentration in the peripheral blood, a local formation in the endometrium can be assumed. A high ehCG concentration is the expression of a physiological function of the endometrium and lack of or a low ehCG concentration is the expression of a pathological function of the endometrium. Since the peripheral blood does not contain tβhCG, a conventional β-hCG-assay (inter alia ELISA, MEIA—see literature citations 18, 19, 21, 22, 23, 24, 28, 29, 30, 54) that does not differentiate between tβhCG and eβhCG can be used for the determination in the peripheral blood alone.

In the prospective or retrospective diagnosis of the receptivity of the mucous membrane of the uterus a differentiation between endometrial and trophoblastic βhCG is not mandatory because there is no pregnancy yet and therefore an expression of tβhCG by a trophoblast is precluded. The invention comprises therefore also the use of a method in which the concentration of total hCG/βhCG or total βhCG is determined for diagnosis of the receptivity of the mucous membrane of the uterus for a fertilized egg. In this method, the determination of hCG is realized preferably with antibodies that recognize αβhCG, the known β7, β6, or β6 hCG subunits. These antibodies must not be specific for eβhCG, i.e., they can also recognize tβhCG.

As such an antibody, a known polyclonal or monoclonal anti αβhCG or an anti βhCG antibody is used preferably. This antibody recognizes preferably an epitope selected from the group of the epitopes β1 to β9 (especially preferred β2 to β8) of the βhCG subunit, of the epitopes cfβ1 to cfβ13 on the βhCG core fragment and the confirmation-dependent epitopes αβ1 to αβ4 of the intact αβ heterodimer according to the classification of the International society of oncodevelopmental Biology and Medicine (ISOBM) (18-20). Such antibodies can be purchased, for example, from the company BIOTREND Chemikalien GmbH, cologne, Germany, or Serotec, Düsseldorf, Germany.

The diagnosis of the receptivity of the mucous membrane of the uterus for a fertilized egg is carried out preferably retrospectively with a sample of menstrual blood or a sample of cells contained in the menstrual blood. For a parallel removal of peripheral blood, the measured hCG value is negligible relative to the hCG in the menstrual blood.

During menstruation, a pregnancy and thus an expression of tβhCG by a trophoblast can be excluded. A differentiation between eβhCG and tβhCG can therefore be carried out in the menstrual blood even without specific eβhCG detection.

The invention therefore also concerns a method for determining defined states or modifications in the mucous membrane of the uterus or in the epithelium of other organs in which the determination of total hCG, βhCG or total hCG/βhCG concentrations is realized, as described above, in a sample of menstrual blood.

Contrary to the generally accepted belief it is not tβhCG that is the first βhCG to be detected in early pregnancy but the endometrial or decidual eβhCG.

Advantageously, the method according to the invention is also suitable to improve the efficacy of already known pregnancy tests. In the known pregnancy tests, only βhCG is determined that is generated by trophoblast or the unspecific total βhCG is determined. Since ehCG is generated already in the well established secretion phase of the endometrium and with the embryo implantation the decidual hCG is released increasingly, it is possible with the method according to the invention to provide a pregnancy diagnosis at an early point in time because, as a result of the early pregnancy occurrence, the ehCG is released into the blood circulation by a flowum instead of being excreted to the exterior as in the case of menstruation.

Since the known tests also cannot provide information as to whether the trophoblast has implanted successfully in the uterus, they often lead to false positive results.

In contrast to known pregnancy tests in which the heterogeneity of the βhCG is not taken into consideration, according to the invention the concentration of endometrial βhCG or non-trophoblastic βhCG is specifically determined. In this way, information is derived in regard to the secretion output and receptivity of the mucous membrane of the uterus which is a prerequisite for a successful pregnancy. The method according to the invention leads therefore to a more reliable pregnancy diagnosis relative to known methods. The method according to the invention enables also the differentiation between an extra-uterine pregnancy or an early pregnancy loss and an intra-uterine pregnancy. In the case of an intra-uterine implantation of the embryo, the expression of eβhCG is higher than in the case of an extra-uterine implantation. The diagnosis of an extra-uterine pregnancy is done preferably by analysis of a serum sample. A low concentration of eβhCG in the serum at normal tβhCG is a sign for extra-uterine pregnancy. In the case of early pregnancy loss eβhCG is present, but tβhCG expression is missing.

Preferably, for pregnancy diagnosis, in addition to the concentration of endometrial βhCG or non-trophoblastic βhCG, also the concentration of trophoblastic βhCG (tβhCG), total βhCG or total hCG is determined also in accordance with the afore mentioned known methods.

The specific determination according to the invention of eβhCG enables for the first time also information whether the βhCG determined by the known methods is indeed tβhCG and how high the proportion of tβhCG and eβhCG is. In this way, it can be reliably determined for the first time whether a pregnancy is actually present or not. A pregnancy is present when thCG has been detected for sure. Since ehCG is of epithelial origin and originates from the woman, a short-term hCG detection according to the prior art after menstruation did not occur is not the same as an early pregnancy. According to the prior art, often early pregnancy losses are misinterpreted as pregnancy. The same holds true for the hCG detection in accordance with the prior art in the second half of the cycle in the case of women wearing a copper IUD. In this case, there is also no pregnancy; instead, the altered endometrium reacts with release of ehCG.

With the differentiated determination of concentration of the ehCG and its relation to thCG it is for the first time possible to differentiate diagnostically whether a pregnancy dysfunction is caused by a change of decidual or the trophoblastic embryonic/fetal unit.

The method according to the invention enables also the possibilities of monitoring the pregnancy during its course and to make a prognosis in regard to possible pregnancy dysfunctions or the success of a pregnancy.

An undisturbed pregnancy is characterized by high ehCG values in the peripheral blood and in the samples of the genital tract (Pap smear, secretion, tissue). When low ehCG values are present there is a risk of miscarriage. By means of the inventive method, the risk of miscarriage can be diagnosed early on and a therapy can be started immediately. Subsequently, the method can be used for therapy control. In this connection, the method according to the invention advantageously enables the differentiation between a dysfunction of the decidua from a trophoblastic/embryonic dysfunction when a miscarriage begins. When a trophoblastic/embryonic dysfunction is present, the tβhCG value is lower.

Other pregnancy dysfunctions such as intra-uterine growth retardation and preeclampsia exhibit non-physiological, i.e., lowered. ehCG values that can be diagnosed with the method according to the invention.

The determination of the ehCG in the serum and in samples of the genital tract (Pap smear, secretion, tissue) can be used advantageously for the screening in regard to premature birth and the determination of the onset of labor. Increased eβhCG values in the secretions of the urogenital tract, in particular, vaginal and cervical secretions, and lowered values in the serum indicate a premature birth or, at the end of pregnancy, the onset of labor.

The specific determination of the ehCG concentration in the amniotic fluid enables advantageously the diagnostics of the decidual function during pregnancy. After birth, the decidual function can be determined advantageously subsequently also by specific determination of the ehCG concentration in the lochia. A high eβhCG concentration is in both cases a sign for a healthy function of the decidua. A reduced tβhCG concentration however is an indication of a pathological pregnancy.

The method according to the invention for specific determination of eβhCG or of non-trophoblastic βhCG also is suitable for determining the effectivity of contraceptive method. Lack of, drop of or temporal displacement of the eβhCG secretion is in this connection a sign of the quality of the contraceptive potency and enables the classification of a method.

For this purpose, the eβhCG concentration in samples (secretion, rinsing liquid, cells, tissue) of the endometrium is determined preferably by ELISA with eβhCG specific antibodies.

When no eβhCG is produced by the endometrium, this is a sign that the endometrium is not receptive for a fertilized egg. Therefore, there is protection against pregnancy.

The method according to the invention can advantageously be used also for differentiation between physiological and pathological epithelium states. In this connection, an epithelial ehCG expression or an expression of non-trophoblastic βhCG (β7, β6) indicates physiological conditions while the detection of t βhCG (β5, β8, β3) in a cell or tissue sample is an indication of a pathological epithelial process, for example, a tumor, a beginning de-differentiation or a beginning carcinogenic degeneration or a carcinoma.

Pathological epithelial processes that can be diagnosed by means of the method according to the invention in particular in this way are endometriosis, myoma, thyroid diseases as well as carcinoma of the endometrium, of the ovaries and of the peritoneum.

The determination of the eβhCG expression and the parallel determination of the total hCG/βhCG or total hCG is carried out in this connection preferably in biological material of the desquamation of the endometrial tissue after separation of the epithelial cells from the stroma cells, the peripheral mononuclear blood cells and the mononuclear immune cells of the endometrial epithelium.

By means of the method according to the invention, it is thus possible to differentiate a proper differentiation of epithelium organs and a false differentiation and beginning carcinogenic degeneration. This is possible because a differentiation is possible between the β7 hCG and the tumor-specific β5 hCG on the level of transcription and translation. The concentrations and their relations of both hCG types provide information in regard to the epithelial health or its dysfunctions in the sense of a de-differentiation. Also, the malignant potency and the prognosis of a tumor disease can be derived therefrom.

Because of the recognition that hGC is also an epithelial hormone that is secreted by the epithelium of the inner surface, the detection of hCG in the serum of healthy non-pregnant female patients and patients without detection of the tumor is not surprising. Presently, this inexplicable hCG detection in female patients/patients without pregnancy and tumor as a so-called phantom hCG is related to the presence of irregular antibodies. By means of the method according to the invention of the specific determination of eβhCG or non-trophoblastic βhCG, these cases of phantom hCG can be explained as a physiological variant of an epithelial functional output. By means of the method according to the invention a physiologically increased expression eβhCG or non-trophoblastic βhCG (β7, β6) can be differentiated from an increased tβhCG (β5, β8, β3) expression in a carcinoma. unnecessary chemotherapeutical treatments and long-term expensive monitoring of these patients are thus obsolete. The determination of concentration of endometrial βhCG (eβhCG) is carried out semi-quantitatively or quantitatively. In a preferred embodiment the concentration determination is realized with at least one antibody that recognizes specifically eβhCG or non-trophoblastic βhCG (tβhCG) by means of an ELISA, a dot-blot, or western blot assay, by an immune-histochemical method, flow cytometry or another known antibody based method. In an alternative embodiment, the determination of concentration of endometrial βhCG (eβhCG) and the differentiation relative to trophoblastic βhCG is done at the level of RNA expression, for example, by known methods of RT-PCR or, for example, by hybridization with an oligonucleotide probe.

The invention comprises also the antibodies which recognize specifically eβhCG and non-tβhCG. The term antibody in the context of the present intention includes, in addition to monoclonal and polyclonal antibodies, also recombinant antibodies and fragments, for example, scFv (single chain fragments) and Fab fragments. Preferably, the antibody has a marker molecule, for example, biotin, dioxygenin, or a fluorescent dye.

The eβhCG-specific antibodies according to the invention recognize preferably a hexa- to deca-peptide in the area of the amino acid position of 117 (SEQ ID No. 1) or in the area of the amino acid positions 2 and 4 (SEQ ID No. 3) of the sequence of the eβhCG (SEQ ID No 10). These eβhCG-specific epitope areas are identified as eβ9 (SEQ ID No. 1) and eβ1 (SEQ ID No. 3). From these epitope areas, the eβhCG-specific antibodies recognize an epitope which comprises amino acid position of 117 or amino acid positions 2 and 4, such as, for example,

```
Pro - Arg - Phe - Gln - Ala - Ser - Ser
                                                117
or
Ser - Arg - Glu - Met - Leu - Arg - Pro -
              2 4
                      3
```

The inventive eβhCG-specific antibodies however do not react with the corresponding tβhCG epitopes β9 and β1 of the corresponding areas (SEQ ID NOS. 2 and 4) of the sequence for tβhCG (SEQ ID No. 8) such as, for example:

```
Pro - Arg - Phe - Gln - Asp - Ser - Ser
                                                117
Ser - Lys - Glu - Pro - Leu - Arg - Pro -
            2 4
```

The inventive antibodies are preferably generated by a peptide selected from the peptide sequence according to SEQ ID No. 1, 12, as well as 3 and 14 or their partial sequences and do not react with the control peptides for tβhCG according to SEQ ID No. 2, 13, as well as 4 and 15.

A further object of the invention is a test kit for determining defined states or modifications in the mucous membrane of the uterus or in the epithelium of other organs.

This test kit comprises at least one antibody that recognizes specifically eβhCG as well as optionally stabilizers, additional antibodies, for example, additional anti-hCG antibodies, secondary antibodies, standards, buffers, reagents for blocking free binding locations (for example, skim milk powder or bovine serum albumin (BSA)).

Preferably, the antibody of the diagnostic kits that recognizes specifically eβhCG is bonded to a solid support. Such a solid support, is, for example, an ELISA carrier, preferably made of polycarbonate, or in the case of a dot-blot or western blot assay a film, preferably made of nitrocellulose.

The bonding of the antibody on the ELISA carrier enables advantageously performing a sandwich ELISA in which bonding of the eβhCG to the eβhCG-specific antibody is detected by a second anti-hCG antibody. The bonding of the antibody to the ELISA carrier is achieved, for example, by incubation of the carrier with an antibody solution in 50 mmol per liter carbonate at a pH value of pH 8 to pH 9 for at least one hour and subsequent drying.

As a second anti-hCG antibody preferably a polyclonal or monoclonal anti-αβhCG or βhCG antibody is used which, in contrast to the inventive eβhCG-specific antibodies, recognizes the endometrial as well as the trophoblastic hCG. This antibody recognizes preferably an epitope selected from the group of epitopes β1 to β9 (preferably β2 to β8) of the βhCG subunit of the epitopes cfβ1 to cfβ13 on the βhCG core fragment and the confirmation-dependent epitopes αβ1to α4 of the intact αβ heterodimer of the classification of the International Society of Oncodevelopmental Biology and Medicine (ISOBM) (18-20). Such antibodies can be purchased, for example, from the company BIOTREND Chemikalien GmbH, Cologne, Germany, or Serotec, Düsseldorf, Germany.

As a standard for eβhCG or as a negative control for tβhCG the test kit contains preferably eβhCG or peptides with an amino acid sequence of 6 to 15 amino acids from the area of the amino acid position 2 and 4 or in the area of the amino acid position of 117 of the sequence of eβhCG (SEQ ID No. 7), i.e., the epitopes eβ1 or eβ9, preferably peptides of the SEQ ID No. 1, 12, 3, 14 or their partial sequences, their solutions.

As a standard for tβhCG or as a negative control for eβhCG the test kit preferably contains tβhCG or peptides with an amino acid sequence of 6 to 15 amino acids of the area of the amino acid position 2 and 4 or in the area of the amino acid position of 117 of the sequence of tβhCG (SEQ ID No. 8), i.e., of the epitopes β1 or β9, preferably peptides of the SEQ ID No. 2, 13, 4, 15 or their partial sequences, their solutions.

A further object of the invention are the isolated endometrial β subunits (eβhCG) of human chorionic gonotrophin (eβhCG) with the amino acids sequence according to SEQ ID No. 10 and the isolated gene sequence for eβhCG according to SEQ ID No. 7 and the use of sequences as marker for the pregnancy diagnosis or for diagnoses of the receptivity of the mucous membrane of the uterus for the fertilized egg. Object of the invention are also the isolated peptide sequences according to SEQ ID No. 1, 3, 12, and 14.

The invention concerns also the use of the eβhCG-specific antibody according to the invention, the isolated peptide sequence according to SEQ ID No. 1, 3, 12 and 14, and the test kit according to the invention for pregnancy diagnosis or for diagnosis of the receptivity of the mucous membrane of the uterus for the fertilized egg.

The invention will be explained with the following embodiments in more detail without being limited to these embodiments.
Embodiment 1: producing a polyclonal antibody specific for the epithelial endometrial hCG molecule (eβhCG) to the βhCG epitope β9 on the C-terminal end (AS 109-123).
Embodiment 2: producing an antibody specific for the epithelial endometrial hCG molecule (e βhCG) to βhCG epitope β1 (AS 1-15).
Embodiment 3: producing monoclonal antibodies.
Embodiment 4: method for detecting eβhCG in bodily liquids and tissue homogenates by means of ELISA.
Embodiment 5: composition of a test kits for detecting eβhCG in body liquids and tissue homogenates by means of ELISA.
Embodiment 6: method for determining optimal implantation conditions by determining endometrial hCG.
Embodiment 7: method for determining physiological endometrium states and for detecting fertilization dysfunctions in the menstrual cycle by determining ehCG.
Embodiments 8: method for retrograde determination of optimal implantation conditions by determining endometrial hCG in the menstrual blood.
Embodiment 9: methods for diagnosis for differentiation between maternal-decidual versus embryonic-trophoblastic dysfunctions in the case of miscarriage tendency and beginning miscarriage.
Embodiment 10: method for premature birth screening or diagnosis of onset of labor.
   Embodiment 1

For obtaining antibodies that recognize specifically endometrial—and decidual-translated eβhCG according to SEQ ID No. 10, in this embodiment the following deca-peptide (SEQ ID No. 12) is used as an antigen with high antigen activity out of the amino acid sequence range (SEQ ID No. 1) recommended in accordance with the invention for the antibody production relative to eβhCG epitope β9:

(SEQ ID No. 12)

P1: Cys - Asp - Asp - Pro - Arg - Phe - Gln -
Ala - Ser - Ser

This P1 is a synthetic peptide with 10 amino acids (AS) from the amino acid sequence 109-123 of the exon 3 of the endometrial variant of the β7 and β6 gene. This peptide differs from the known epitope β9 near the C- terminal peptide (CTP-βhCG) of the trophoblastic βhCG subunit by one alanine (Ala)—instead of aspartate (Asp)—at position 8 of the peptide (amino acid position 117 in βhCG). The selected amino acid sequence differs considerably from the sequence of the βLH subunit (SEQ ID No. 11). Alternatively, other peptides with 7 to 15 amino acids out of the sequence area of the amino acid sequence area eβhCG epitope eβ9 (SEQ ID No. 1) can be used that contain alanine (Ala) at the position that corresponds to the amino acid position 117 in eβhCG (SEQ ID No. 10).

For obtaining control antibodies that recognize specifically the β5, β8, β3 subunits of the known trophoblastic hCG (thCG) (SEQ ID No. 8), in this embodiment the following synthetic deca-peptide (SEQ ID No. 13) is used as an antigen of high antigen activity out of the amino acid sequence area (SEQ ID No. 2) in the epitope β9 near the CTP-βhCG recommended in accordance with the present invention relative to βhCG epitope β9:

(SEQ ID No. 13)

K1: Cys - Asp - Asp - Pro - Arg - Phe - Gln -
Asp - Ser - Ser

The peptides P1 and K1 were produced by conventional solid-phase peptide synthesis, purified by gel filtration and ion exchange chromatography and specified by HPLC (23, 49).

The peptides P1 and K1 selected in accordance with the present invention from the amino acid sequence area for the βhCG epitope β9 in this embodiment are bonded by means of the EZ Antibody Production and Purification Kit, carboxylate reactive (Pierce chemical Co., Rockford, Ill.) according to manufacturer=s recommendations to the protein keyhole limpet hemocynain (KLH) as a carrier (53). In accordance with the recommended procedure, the peptides were bonded to bovine serum albumin (BSA) as a carrier for the subsequent ELISA. For producing polyclonal antibodies, five 12-week old rabbits (New Zealand white, each approximately having a weight of 2 kilograms) are each injected with different solutions of a total volume of 500 μl i.p. Rabbit #1 receives 200 μg KLH-bonded peptide P1 in 0.1% Nacl with 1:1 adjuvant Specol (ID-DLO, Lelystad); rabbit #2 receives 500 μg KLH-bonded peptide K1in 0.1% NaCl with 1:1adjuvant Specol; rabbit # 3 receives only 0.1% NaCl with 1:1adjuvant Specol. After 14 days, the injection is repeated with the same solution i. p. (booster shots), respectively. The last booster shot was applied three weeks after the first booster shot with the same solution. 14 days after the last booster shot the serum was removed, respectively. The sera were examined by ELISA according to standard conditions in regard to the presence of specific antibodies against P1.

For ELISA, the peptide P1 bonded to BSA was first applied in a concentration of 10 μg/ml in a coating buffer (0.1 mol/liter sodium carbonate/bicarbonate, pH 9.6) in 50 μl per well onto a Maxisorp ELISA carrier (Nunc) and incubated for one hour at 37 ▭C. Parallel to this, a carrier (=negative control carrier) was coated accordingly with a solution of the BSA-bonded control peptide K1. The wells were subsequently washed five times with phosphate-buffered sodium chloride solution with addition of 0.1% Tween 20 (PBS-T). Subsequently, 200 μl of a 10% milk powder solution in PBS-T was added, respectively, and incubated for one hour at 37 ▭C and washed once with PBS-T. The thus prepared carriers were incubated with the immune sera, washed three times with PBS-T, and incubated for 0.5 h with biotinylated anti-rabbit-IgG (Dako) as a secondary antibody and washed three times with PBS-T.

subsequently, a 1:2,000 dilution of a streptavidin-peroxidase conjugate (sigma) in PBS-T was added. After 30 minute incubation at 37 ▭C the carrier was washed twice with PBS-T and once with PBS and a substrate (100 μl ) containing o-phenylene diamine hydrochloride was added. The yellow-brown color development was stopped after 5 minutes by adding 50 μl 2M H₂SO₄ and the optical density was determined at a wavelength of 490 nm (reference wavelength: 650 nm). P1-specific antibodies could be detected only in rabbit #1 in the serum that were obtained seven days after injection but not in the serum before the injection as well as in none of the serum of rabbits #2 and #3. These experiments demonstrate that the peptide P1 is able to induce specific antibodies in rabbits.

For immunoaffinity chromatographic purification of the antibodies from the serum the peptide p1 is immobilized in accordance with manufacturer=s procedures for EZJ Antibody Production and Purification Kits, carboxy Reactive (Pierce) to a diamino dipropyl amine column (53). The serum, from which the specific antibodies were to be purified, was incubated for inactivation for 30 minutes at 56 ▭c. Subsequently, it was applied to the column and allowed to pass. Subsequently, the column was washed with 20 ml PBS and with 10 ml of a 0.5 mol/liter MgCl₂ solution. The specifically bonded antibodies were washed out by addition of 3 ml 3 mol/liter MgCl₂ followed by 3 ml of 4 mol/liter MgCl₂. The washed-out liquids were filled into dialysis hoses (Pierce) and dialyzed overnight at 4 ▭C against one liter of PBS. Subsequently, the protein content of the dialyzed preparations was determined by means of BCA protein kits (Pierce), the purity was determined by SDS-PAGE and coomassie blue coloration as well as the specificity of the antibody preparation by means of ELISA.

### Embodiment 2

For the further amino acid differentiation between tβhCG and eβhCG in the βhCG epitope section β1 at the amino acid positions +2 (Lys to Arg) and +4 (Pro to Met), an eβhCG specific antibody and a tβhCG specific control antibody are produced.

For generating antibodies that recognize specifically the endometrial and decidual translated eβhCG according to SEQ ID No. 10, in this embodiment the following synthetic peptide (SEQ ID No. 14) as antigen with high antigenicity is used from the amino acid sequence area (SEQ ID No. 3) recommended for the antibody preparation in accordance with the invention relative to the βhCG epitope eβ1:

(SEQ ID No. 14)

P2: Ser - Arg - Glu - Met - Leu - Arg - Pro -
Arg - Cys - Arg - Pro

This P2 is a synthetic peptide of the amino acid sequence area 1-15 in exon 2 that differs from the known epitope β1 of the trophoblastic βhCG subunit in regard to two amino acid positions. The selected amino acid sequence P2 difference in this epitope section also significantly from the sequence of the βLH subunit (SEQ ID No. 11). Alternatively, other peptides with 7 to 15 amino acids of the sequence area of the amino acid sequence range eβhCG epitope eβ1 (SEQ ID No. 3) can be used which have argenin (Arg) and methionin (Met) at the positions corresponding to the amino acid positions 2 and 4 in eβhCG (SEQ ID No. 10).

For obtaining control antibodies that recognize specifically tβhCG (β5, β8, β3), the same procedure in accordance with the adequate peptide (SEQ ID No. 15) selected as an example of the amino acid sequence of the βhCG gene β5 of the epitope β1 (SEQ ID No. 4)—amino acid sequence area AS 1 to 15 of the t=hCG (SEQ ID No. 8) is carried out:

(SEQ ID No. 15)

K2: Ser - Lys - Glu - Pro - Leu - Arg - Pro- Arg -
Cys - Arg - Pro

The immunization of the rabbits was equally successful with P2 and K2 as in the case of P1 and K1. The eβhCG-specific antibodies were also obtained with P2 that showed no cross-reactivity for K2 or tβhCG and also no cross-reactivity with βLH.

### Embodiment 3

Producing monoclonal antibodies (mAb) is carried out in accordance with the hybridoma preparation well described in the literature for ISOBM-MAb h54, 264, 277, 278, 287, 282, and 313 for epitope β8, FB-12 and 280 for epitope β9, and 256, 274, and 284 for epitope β1 with carrier-bonded synthetic βhCG peptide sequences (18, 20-24, 45-48), wherein in the peptide sequences the amino acids at position +2, +4, and +117 were exchanged for the endometrial hCG (eβhCG). The preparation of ISOBM-mAb showed that the preparation of hybridoma which secret antibodies of the desired βhCG specificity can be repeated reliably (18, 24, 45-48).

For hybridoma preparation, the carrier-bonded synthetic eβhCG specific peptide sequences P1 and P2 (SEQ ID NOS. 12 and 14) as well as the control peptides K1 and K2 (SEQ ID No. 13 and 15) were used.

The preparation of the monoclonal antibodies for P1 will be explained in the following in more detail:

The immunization was realized in BALB/C mice. For this purpose, for each mouse approximately 1 mg of purified carrier-bonded peptide was required.

Immunization: eight female BALB/C mice (Roche, Institut fur Biologisch-Medizinische Forschung, Basel, Switzerland), six to eight weeks old, are immunized by means of the KLH-bonded peptide P1 produced in accordance with embodiment 1 for each animal according to the following protocol (18, 20, 24, 45-51): The first immunization was realized by subcutaneous injection of 50-150 μg βhCG peptide immunogen on the carrier for each animal in complete Freund=s adjuvant. The further immunizations were carried out every other day by injection of the same amount of immunogen in incomplete Freud=s adjuvant. On day 17 the mice received an intraperetonal immunization again with 50-150 μg of the antigen in PBS support for each animal, respectively. The immun sera are tested with the hCG-POD system in regard to released antibodies (ELISA in embodiment 1). The mice with the high hCG antibody immune responses (approximately 3) are boosted again with 50-150 μg βhCG immunogen and were assigned for fusion after three days.

Fusion: after immunization from the knee joint lymph nodes and spleen of mice immunized in this way the splenocytes (B-lymphocytes) are isolated and fused with cells of the mouse myeloma cell line P3-x63-Ag8.653 (American Type culture collection) according to the method of Köhler and Milestein (51) as described in Kovalevska (47). The ratio of splenocytes to myeloma cells in this connection is 4:1 to 6:1. RPMI-1640 with 10% fetal calf serum (FCS) or polyethylene glycol 1500 (sigma) is used as a fusion medium. The immun serum of the selected mice is collected as positive control.

Selection of fused cells (hybridoma cells): The generated hybridoma cells after fusion are separated from the myeloma cells that have not undergone fusion, are distributed onto microtiter carriers and, together with peritoneal ascites cells of the mouse are cultivated for a week in a RPMI culture medium that contains hypoxanthine, aminopterin, and thymidine (HAT) with 10% FCS (46-50). Half of the medium was replaced every three days. On the days 12-14 after fusion a portion of the liquid above the culture of the wells was tested in regard to the presence of hCG antibodies by ELISA (screaming of the oligoclones).

The screening of the oligoclones was realized by ELISA as described in embodiment 1 with the difference that in place of the biotinylated anti-rabbit IgG a biotinylated anti-mouse IgG antibody (Dako) was used.

The liquid above the culture of 10% of the screened wells show with ELISA carrier on which P1 was immobilized but not with the negative control carrier, a color reaction in ELISA. Thus, 10% of the obtained oligoclones recognize therefore specifically the peptide P1 but not K1.

Three especially productive Ig-positive cell clones, P1.1, P1.2, and P1.3, show an especially high antibody titer with the desired ehCG specificity and were selected for the further subcloning.

Subcloning: The selected oligoclones are now subcloned to monoclonality (50). In this connection, positive clones were selected and multiplied in vitro (cloning by limiting dilution method). The isolated colonies were tested again with ELISA. The positive-testing clones were used for the next cycle of cloning. Three cycles of cloning are required in order to obtain specific stable clones. They were used for the formation of 100 ml liquid with the monoclonal antibodies.

The molecular hCG antibodies were subsequently purified by affinity chromatography with the protein A-sepharose purification system for monoclonal antibodies (Biorad). The purity of mAb was tested by SDS polyacrylamide gel electrophoresis and subsequently the protein concentration was determined (18, 52). The specificity of the antibodies was assayed against the heterodimer αβhCG molecule with eβhCG as a β subunit as well as against the indicated peptide P1 in the above described ELISA.

The monoclonal antibodies produced in this way by immunization, isolation, hybridization, and purification are stored at −20 ▭C.

The production of monoclonal antibodies with the peptide P2 was carried out in accordance with the same procedure and lead to comparable results. With P2 eβhCG specific antibodies were also obtained that did not exhibit cross-reactivity for K2 or tβhCG.

### Embodiment 4

For detecting the epithelial endometrial and decidual hCG (ehCG) in body liquids and tissue homogenate, the endometrial or decidual βhCG antibodies that are specific for βhCG epitope β8 and βhCG epitope β1 and prepared in accordance to embodiments 1 to 3 are adsorbed on microtiter plates and test systems on the basis of ELISA technology are developed. Adequate ELISA arrangements are used as a control system by employing comparable trophoblastic βhCG (thCG) antibodies of the respective βhCG epitopes β1 and β8 (18, 21-24, 47).

Sandwich ELISA: In accordance with embodiment 3 the immune-purified monoclonal eβhCG antibodies specific to P1 and P2 (eβhCG) as well as K1 and K2 (tβhCG) are adsorbed as initial antibodies in a solution of 100 μl/well of Maxisorp ELISA carriers (Nunc, 96 wells) (10 μg/ml in 200 mM bicarbonate buffer, pH 9.6, 1 hour at 37 ▭C or overnight at 4 ▭c). The wells are subsequently washed twice with washing buffer (10 mM PBS, pH 7.2 with 0.05% Tween 20) and incubated for one-hour with blocking buffer (1% BSA in PBS pH 7.2).

Subsequently, incubation (100 μl, one hour, 37 ▭C, respectively) with the serum in which the endometrial hCG (eβhCG) is to be assayed. The serum is diluted for this purpose 1:10 to 1:1,000 in blocking buffer. As a standard series of hCG determination additionally the synthetic peptides P1, P2 (specific to endometrium) and K1 and K2 (specific to trophoblast) are incubated in six different concentration stages between 0 and 1000 ng/ml.

When hCG is present in the employed samples, it will bind on the immobilized endometrial or trophoblastic specific antibodies of the wells. The assay system employs after respective washing steps the second biotinylated anti-βhCG-antibody that binds as a sandwich to the immobilized solid phase βhCG antibody/βhCG complex. As a second monoclonal hCG/βhCG antibody that recognizes the endometrial as well as the trophoblastic total molecule hCG and its β-subunit, in this embodiment a biotinylated antibody specific for the hCG β2 epitope (INN-22 Serotoc) is used. After incubation at room temperature and additional washing steps for removing excess enzyme-bonded βhCG antibody, the same process as in embodiment 1 is carried out. The detection is initiated with a 1:2,000 dilution of the streptavidin preoxydase conjugate (Sigma) in PBS-T.

After incubation for 30 minutes at 37 ▭C, the carrier was washed twice with PBS-T and once with PBS and a substrate (100 μl) containing o-phenylene diamino hydrochloride was added. The yellow-brown color development was stopped after five minutes by adding 50 μl 2 M sulfuric acid and the optical density was determined at a wavelength of 490 nm (reference wavelength: 650 nm).

### Embodiment 5

A test kit for specific detection of endometrial respectively decidual hCG and its βhCG subunit (eβhCG) in body liquids and tissue homogenates by ELISA contains, for example, the following components:
1. an ELISA carrier (10 μg per well, respectively) that is precoated with eβhCG-specific antibody clone P1.2 of embodiment 3 (specific for eβhCG expressed in the endometrium and decidua; does not recognize t βhCG);
2. six dilutions of the peptide P1 as a standard series (0, 10, 50,100, 500, 1,000 μg/ml);
3. washing buffer PBS-T (10 mM PBS, pH 7.2 with 0.05% Tween 20);
4. blocking buffer (1% BSA in PBS, pH 7.2); 5. biotinylated total hCG/βhCG antibody as second hCG antibody specific for the hCG β2 epitope (INN-22 Serotec);
6. streptavidin-HPR conjugate (Dako);
7. PBS (Dako);
8. o-phenylene diamine as substrate;
9. 2 M sulfuric acid as a stop solution.

As an alternative to component 5, the test kit contains, for example, as a second hCG antibody a biotinylated antibody specific for the hCG β4 epitope (INN-24, Serotec).

An adequate test kit as a control kit or for specific determination of the trophoblastic hCG in body liquids or homogenized tissue samples contains, for example, the above-mentioned components 3 to 9, and, instead of component 1, a tβhCG-specific antibody obtained by immunization with K1, and, instead of component 2, appropriate dilutions of the control peptide K1 as a standard series.

with the kit for specific detection of the eβhCG the quantification and evaluation of specific epithelial endometrial hCG secretion (e βhCG) in body liquids and cell or tissue homogenates of the early to middle secretion phase of the menstrual cycle, optimal implantation conditions (embodiment 6) as well as possible fertilization dysfunctions (embodiment 7) can be detected—prospective as well as retrograde—in consideration of endometrial diagnostics and therapy control.

### Embodiment 6

For the prospective diagnostics of embryo receptivity in the early secretion phase of the current cycle from the female patient wanting a child, for example, at the middle of the cycle a Pap smear with cervical secretion of the cervical channel or a vaginal Pap smear with vagina secretion is taken for diagnostic evaluation of implantation conditions. This Pap smear is examined with regard to the present beginning or already ongoing expression or secretion oβhCG by means of the ELISA disclosed in embodiment 4. In this way, it is also possible to provide information in regard to the quality of the secretory transformation and the expected receptivy of the endometrium.

In the context of in-vitro fertilization two days after follicle puncture a cotton swab is shortly inserted into the cervix or the vaginal pap smear is taken and by means of ELISA disclosed in connection with embodiment 4 the activation of eβhCG is diagnosed. A positive eβhCG result signalizes a receptive endometrium and the embryo still being cultured can be transferred one or two days later. Should the test be negative, the embryo will be cryo-preserved and will be flushed into the uterine cavity during the next cycle that was determined to be positive for eβhCG. In this way, decisions in regard to embryo transfer or insemination of hormonally stimulated female patients for the actual or the subsequent cycle can be made. In addition to the cervical secretion it is also possible to employ sample material (tissue, cells, perfust) of other epithelial organs such as the oral mucous membrane or vaginal mucous membrane. Since all epithelial organs are subject to the cycle more or less they can also be incorporated into the examination.

### Embodiment 7

With this embodiment, in the female patients physiological or pathological endometrium states can be detected and possible fertilization dysfunctions under the aspect of endometrial hCG diagnostics and therapy control can be detected and evaluated during the actual cycle or the subsequent cycle.

For this purpose, a Pap smear of the cervical channel with cervical secretion or a vaginal Pap smear with vaginal secretion is taken from the female patient for diagnostic evaluation during the secretory phase of the endometrial transformation, primarily during the middle secretion phase about the 20th to 24th day of the cycle.

This Pap smear is examined with regard to the present beginning or ongoing expression or secretion of eβhCG by means of ELISA described in embodiment 4. In this way, information in regard to the lack of, low-value or high secretory transformation of the endometrium and the quality of the expected receptivity of the endometrium can be made for the female patient.

The presence of unequivocally measurable eβhCG on the 20th to 24th day of the cycle signalizes a healthy endometrium transformed properly with regard to time and function. At the same time, this is an expression of unhindered interaction between hypothalamus and pituitary gland, the ovaries and the uterus. With the presented method of a Pap smear of patients for examination of the ehCG in body liquids and cell and tissue homogenates, the diagnostics and therapy control of the uterus function can be performed. For this ehCG determinations with the above-mentioned methods, it is also possible to employ the endometrium sampled by curettage according to diagnostic indication.

### Embodiment 8

The removal of menstrual blood from female patients after un-stimulated, stimulated or disturbed cycle represents an important, simple and up to now unused method for retrograde implantation diagnostics in order to detect information for the secretory transformation of the endometrium of the previous cycle and optionally information for the receptivity of the subsequent cycle. This non-invasive method can supplement or replace the invasive method of diagnostic sample curettage with regard to its information contents.

The menstrual blood as a result of the endometrial desquamation is used like peripheral blood for separation of cell material and stroma for the direct measurements of endometrial hCG secretion (ehCG ) with specific ehCG antibodies in an ELISA test in accordance with embodiment 4. The menstrual blood is taken after a spontaneous cycle, after hormone therapy, after IVF and ET without successful implantation as well as for proposed diagnostics of the cycle in the case of females wanting children and in the case of IUD patients, myoma and endometriosis. In this connection, a parallel analysis of peripheral blood is provided at the same time for exclusion of an also increased serum value of ehCG.

The epithelial and stroma cell material of the endometrial tissue after desquamation that has been separated from the menstrual blood is used like the menstrual plasma by means of ELISA tests for evaluating the expression and secretion of endometrial hCG in the preceding cycle.

### Embodiments 9

The diagnostics for a differentiation between maternal-decidual versus embryonal-trophoblastic dysfunctions in the case of miscarriage tendency and beginning of miscarriage is based on the fact that eβhCG is expressed in the maternal-decidual tissue of pregnancy as in the secretory endometrium. In the embryonal-trophoblastic tissue of the placenta trophoblastic hCG is expressed and translated. The hCG concentrations of the peripheral blood in the pregnancy show a secretion maximum in the first trimester and are significant for the second and third trimesters.

In this embodiment, an application is demonstrated in which hGc differentiated during pregnancy as endometrial/decidual hCG (ehCG) and trophoblastic hCG (ThCG) can be detected in peripheral blood but also vaginal or cervical secretions of a Pap smear, in the released amniotic fluid in the case of perforated or burst amniotic sac, in the lochia blood as well as in other epithelial secretions and/or their cell and tissue homogenates by ELISA or in quantitative real-time RT-PCR.

In the case of an impending miscarriage, characterized by the beginning of uterine bleeding, a therapeutic poly-pragmatic approach is used because first the reason of the dysfunction is unclear. In the case of an impending miscarriage by differentiation of embryonic hCG (ehCG) and the trophoblastic hCG (thCG) it is possible to differentiate between a maternal-decidual versus trophoblastic-embryonic cause of the dysfunction.

For this purpose, serum of the patient or the above mentioned other bodily liquids and cell and tissue homogenates are taken and examined by means of the ELISA kits developed with the present invention in regard to ehCG and/or thCG (embodiment 4). It is also possible to detect thCG by a conventional commercially available kits that are recommended for common placenta hCG measurements during pregnancy (DPI, Abbott, Serono, Roche, Baxter). A low ehCG value indicates a beginning decidual insufficiency while a comparatively low thCG value signalizes a dysfunction of the fetus/placenta unit. While the first dysfunction can be treated, in the case of the dysfunction of the thCG secretion it must be determined whether a therapy is possible and expedient.

Also, the prognosis of an impending miscarriage can be detected in the blood. For this purpose, miscarriage blood is taken with a speculum from the rear vaginal cavity and a determination of ehCG with the ELISA kit according to the invention is performed. In the case of minimal bleeding, blood or bloody cervix mucus can be taken by a cotton swap and can be tested by ELISA test (embodiment 4) and/or quantitative real-time RT-PCR with regard to eβhCG. A high ehCG result indicates a decidual dysfunction. In the case of very high ehCG values, a miscarriage that cannot be treated is to be expected.

The method of this embodiment can also be used as a therapy control in the treatment of decidual dysfunctions.

### Embodiment 10

Screening for premature delivery or diagnostics of the onset of labor is based on that the embryonic hCG (ehCG) in the genital tract is increased before premature birth while ehCG in the serum is lowered. For this reason, the above-mentioned method can be used for screening for premature birth. The same pattern is also found at the beginning of the birthing process at the end of the pregnancy.

For premature birth screening or for diagnosis of beginning of the birth, secretions or cells are removed from the genital tract (cervix, rear vaginal cavity) of the female patients. In the secretion, ehCG is determined with the above-mentioned ELISA test; in the case of removal of cells, the ehCG β7 expression is determined in the secretion by means of quantitative real-time RT-PCR. An increased ehCG result in the secretion like a reduced ehCG β7 in the cells of the genital tract signals the beginning of a premature birth.

Moreover, premature birth screening is also possible a serum examination. In this connection, serum of the patient is obtained and ehCG is determined. At low or dropping ehCG values, a premature birth is to be expected.

Instead of the ELISA test described in the embodiments, it is also possible to employ a quantitative detection of the ehCG gene expression by real-time RT-PCR for detecting the eβhCG concentration in the tissue samples.

List of Abbreviations:

In the description the following abbreviations are being used:
αhCG alpha-subunit of human chorionic gonadotrophin
βhCG beta-subunit of human chorionic gonadotrophin
BSA bovine serum albumin
CTP C-terminal peptide
EDTA ethylene diamine tetra acetate
ET embryo transfer
EβhCG endometrial beta-subunit of human chorionicic gonadotrophin
ehCG endometrial human chorionicic gonadotrophin (eβhCG +αhCG)
hCG human chorionic gonadotrophin
IgG immunoglobulin gamma
ISOBM International society of oncodevelopmental Biology and Medicine
IUD intrauterine device
IVF in-vitro fertilization
KLH keyhole limpet hemocyanin hemocyanin
PBS-T phosphate buffered saline solution with 0.1 % Tween 20 added
ELISA enzyme-linked immunosorbent assay
mAb monoclonal antibody
MEIA microparticle enzyme immunoassay
mM mMol/liter
PBS phosphate buffered amine solution
tβCG throphoblastic beta-subunit of human chorionicic gonadotropin
   cited Non-Patent Literature:
(1) J. C. Pierce, T. F. Parsons, Annu. Rev. Biochem., 50 (1981) 465-495
(2) P. A. Rothman, V. A. Chao, M. R. Taylor et al., Mol. Reprod. Dev., 33 (1992) 1-6
(3) S. Dirnhofer, M. Hermann, et al., J. Clin. Endocrinol. Metab., 81 (1996) 4212-4217
(4) Z. M. Lei, P. Toth, C. V. Rao und D. Pridham, J. Clin. Endocrinol. Metab.,77 (1993) 863-972
(5) T. Yokotani, T. Koizumi, R. Taniguchi et al., Int. J. Cancer, 71 (1997) 539-544
(6) P. Berger, W. Kranewitter, S. Madersbacher et al., FEBS Lett., 343 (1994) 229-233
(7) D. Bellet, V. Lazar, I. Bieche et al., Cancer Res., 57 (1997) 516- 523
(8) I. Marcilliac, F. Troalen, J. M. Bidart et al., Cancer Res., 52 (1992) 3901-3907
(9) H. Alfthan, C. Haglund, J. Dabek et al., Clin. chem., 38 (1992) 1981-1987
(10) V. Lazar, S. G. Diez, A. Laurent et al., Cancer Res., 55 (1995) 3735-3738
(11) P. N. Span, C. M. G. Thomas, J. J. Heuvel et al., J. Endocrino7., 172 (2002) 489-495
(12) M. Lundin, S. Nordling, J. Lundin et al., Int. J. Cancer, 95 (2001) 18-22
(13) K. Hotakainen, B. Ljungberg, A. Paju et al., Brit. J. Cancer, 86 (2001) 185-189
(14) D. S. Hoon, T. Sarantou, F. Doiet al., Int. J. Cancer, 69 (1996) 369-374
(15) M. Bo und I. J. Boime, J. Biol. Chem., 267 (1992) 3179-3184
(16) K. Talmadge, N. C. Vamvakopoulus und J. C. Fiddes, Nature, 307 1984) 37-40
(17) P. Policastro, C. Ovitt, M. Hoshinaet al., J. Biol. Chem., 258 (1983) 11492-11499
(18) P. Berger, C. Sturgeon, J. M. Bidart et al., Tumor Biol., 23 (2001) 1-38(19) J. M. Bidart, S. Birken, P. Berger et al., scand. J. Clin. Lab. Invest., 216 (1993) 118-136
(20) P. Berger, J. M. Bidart, P. S. Delves et al., Mol. Cell Endocrinol., 125 (1996) 33-43
(21) S. Dirnhofer, S. Madersbacher, J. M. Bidart et al., J. Endocrinol., 141 (1994) 153-162
(22) P. Berger, R. Klieber, W. Panmuong et al., J. Endocrinol., 125 (1990) 301-309
(23) J. M. Bidart, F. Troalen, C. J. Bohuon et al., J. Biol. Chem., 262 (1987) 15483-15489
(24) J. M. Bidart, D. H. Bellet, G. F. Alberici et al., Mol. Immunol., 24 (1987) 339-345
(25) P. K. Hotakainen, E. M. Serlachius et al., Mol. Cell. Endocrinol., 162 (2000) 79-85
(26) A. K. Miller-Lindholm, C. J. Labenz, J. Ramey et al., Endocrinology, 138 (1997) 5459-5465
(27) S. Madersbacher, C. Kratzik, R. Gerth et al., Cancer Res., 54 (1994) 5096-5100
(28) R. Oyasu, L. Nan, P. Smith et al., Arch. Pathol. Lab. Med., 119 (1994) 715-717
(29) L. A. Cole, Clin. Chem., 43 (1997) 2233-2243
(30) L. A. Cole, J. Reprod. Med., 43 (1998) 3-10
(31) L. A. Cole, K. M. Rinne, S. Shahabi et al., Clin. Chem., 45 (1999) 313-314
(32) A. Kardana, M. M. Elliot, M. A. Gawinowicz et al., Endocrinology, 129 (1991) 1541-1550
(33) S. Birken, A. Krichevsky, J. O° Connor et al., Endocrine, 10 (1999) 137-144
(34) A. Krichevsky, S. Birken, J. O'Connor et al., Endocrinology, 134 (1994) 1139-1145
(35) G. Kovalevskaya, S. Birken, T. Kakuma et al., J. Endocrinol., 161 (2000) 99-109
(36) P. Mock, G. Kovalevskaya, J. F. O'Connor et al., Hum. Reprod., 15 (2000) 2209-2214
(37) P. Mock, P. Bischof, R. Rivest et al., Hum. Reprod., 13 (1998) 2629-2632
(38) M.Seppälä, E.-M. Rutanen, et al., J. Clin. Endocrinol. Metab., 47 (1978) 1216-1219
(39) R. W. Sharpe, W. Wrixon, et al., J. Clin. Endocrinol. Metab. ,45 (1977) 496-499
(45) G. Kovalevskaya, S. Birken, T. Kakuma et al., Clin. Chem., 45 (1999) 68-77
(46) C. Stähli, T. Staehelin, V. Miggiano et al., J. Immunol. Method., 32 (1980) 297-304
(47) G. Kovalevskaya, S. Birke, J. O=Connor et al., Endocrine, 3 (1995) 881-887
(48) J. B. Conde, A. B. Moreno, C. A. Bas, Hybridoma and Hybridomics 21 (2002) 381-384
(49) R. B. Marrifield, J. Am. Chem. Soc., 85 (1963) 2149-2154
(50) A. M. Krieg, Am. Rev. Immunol., 20 (2002) 709-760
(51) G. Köhler, C. Milestein, Nature, 256 (1975) 495
(52) S. Madersbach, P. Berger, Methods, 21 (2000) 41-50
(53) Pierce, Rockford, USA: EZ Antibody Production and Purification Kit, Carboxy Reactive
(54) L. A. Cole, Gynecol. Oncol., 71 (1998) 325-329

What is claimed is:

1.-21. (canceled)

22. A method for determining defined states or modifications in the mucous membrane of the uterus or in the epithelium of other organs, the method comprising the step of:
determining specifically a concentration of at least one of human endometrial chorionic gonadotropin (eβhCG/ehCG) and non-trophoblastic hCG (hCG type I, β6, β7) in a sample of at least one of body liquid, tissue, and cells.

23. The method according to claim 22, further comprising the step of determining a concentration of trophoblastic hCG (hCG type II, tβhCG) or total βhCG or total hCG.

24. The method according to claim 22, wherein in the step of determining the concentration of endometrial hCG (eβhCG/ehCG) at least one antibody that recognizes specifically endometrial hCG (eβhCG/ehCG) and does not recognize trophoblastic hCG (hCG type II, tβhCG) is used.

25. The method according to claim 24, wherein the at least one antibody recognizes specifically a peptide selected from peptide sequences according to SEQ ID No. 1 or 3 or partial sequences thereof.

26. The method according to claim 23, wherein the concentration of endometrial hCG and optionally trophoblastic hCG or total βhCG or total hCG is determined in a sample selected from secretions, perfusion liquid, cells or tissue, wherein the sample originates from peripheral blood, serum, lochia, menstrual blood, amniotic fluid, urine, saliva, eye chamber fluid, the urogenital tract, the gastrointestinal tract, the respiratory tract or the central nervous system.

27. The method according to claim 22 for determining receptivity of the mucous membrane of the uterus for a fertilized egg in prospective and retrospective embryo implantation diagnostics, comprising the step of taking a sample in the early luteal phase in the form of tissue from the endometrium or from the cervical mucous membrane, a secretion of the vagina, the cervix, or the uterus, or serum, plasma, or peripheral blood and determining in the sample the non-trophoblastic or endometrial βhCG concentration.

28. A method for determining defined states or modifications in the mucous membrane of the uterus or in the epithelium of other organs, the method comprising the step of determining a concentration of total hCG or of β subunits thereof in a sample of menstrual blood.

29. An antibody recognizing specifically endometrial hCG (eβhCG/ehCG) and not trophoblastic hCG(hCG type II, tβhCG) and recognizing specifically a peptide selected from the peptide sequences according to SEQ ID No. 1 or No. 3 or partial sequences thereof.

30. An antibody recognizing specifically the trophoblastic human chorionic gonadotropin (hCG type II/tβhCG) and not endometrial human chorionic gonadotropin (e βhCG/ehCG) and recognizing specifically a peptide selected from the peptide sequences according to SEQ ID No. 2 or No. 4 or partial sequences thereof.

31. A test kit for determining defined states or modifications in the mucous membrane of the uterus or in the epithelium of other organs comprising at least one antibody according to claim 29 or 30 and further antibodies and standards.

32. An andometrial β subunit of human chorionic gonadotropin (eβhCG) having an amino acid sequence according to SEQ ID No. 10.

33. A gene sequence β6e coding for the endometrial β subunit of human chorionic gonadotropin (eβhCG) according to SEQ ID No. 7.

34. A peptide selected from the amino acid sequences according to a SEQ ID No. 1, 3, 12, and 14.
